(19) United States
(12) Patent Application Publication
(10) Pub. No.: US 2007/0237811 A1
(43) Pub. Date: Oct. 11, 2007

(54) Chitosan wound dressing

(76) Inventor: George H. Scherr, Park Forest, IL (US)

Correspondence Address: George H. Scherr, Ph.D., 33 Monee Road, Park Forest, IL, 60466, US

(21) Appl. No.: 11/400,822

(22) Filed: Apr. 10, 2006

(51) Int. Cl. A61L 15/00; A61K 31/722

(52) U.S. Cl. 424/445; 442/123; 514/55

(57) ABSTRACT

A composition is described in which chitosan is prepared in a foamed gel that may be layered onto a suitable backing for use as a wound dressing, or the gel may be directly applied to wounds to effect hemostatic activity as a result of the action of the chitosan. The composition of the foamed chitosan gel has added to it medicaments, which include antimicrobial agents, in order to reduce the risk of microbial infections in wounds where hemostatic agents including the chitosan foamed gel preparation can be applied to effect hemostasis.

### BACKGROUND OF THE INVENTION AND DESCRIPTION OF PRIOR ART

Methods to develop hemostatic dressings have been pursued for many years. Oxidized cellulose was a hemostatic dressing that was prepared from cellulosic products and had reasonably good hemostatic activity.

Collagen, either extracted as porcine collagen or bovine collagen, has been used as a hemostatic dressing.

Chitin is a polysaccharide that can be extracted from shellfish, clams, oysters, and other organisms. The deacetylation of chitin results in chitosan in which the —NHCO—CH₃ group of the chitin has been replaced with an acetamide group.

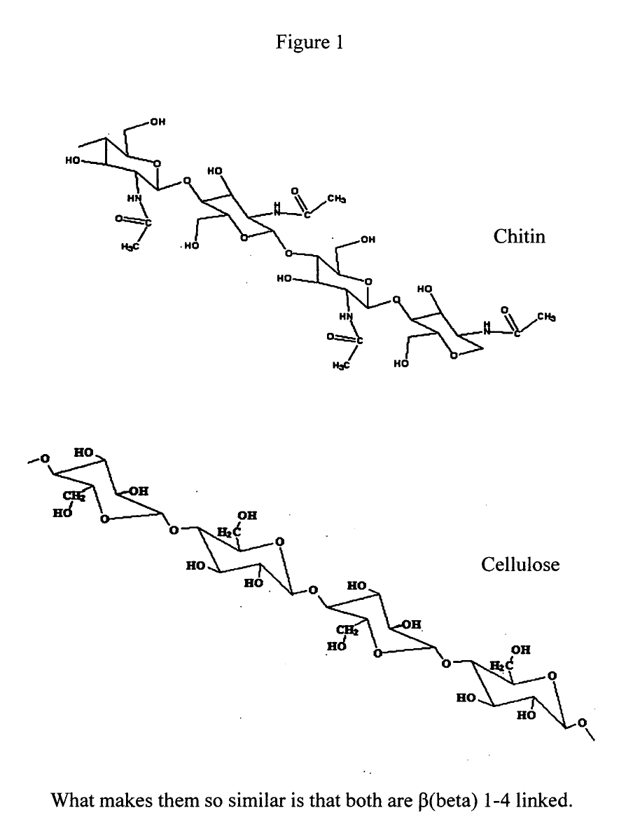

The molecular structure of chitin and cellulose are very similar and it would be expected that chitin and/or chitosan would be amenable for being introduced into the same composition as other cellulosic molecules, such as alginate (see FIG. 1).

Experiments performed as early as the 1950 have shown that chitosan is an effective cellular agglutinating agent. As a result of such early studies, it was shown that chitosan can agglutinate red blood cells which agglutination results from the high negative charge on the cell membranes of the red blood cells and the net positive charge of the chitosan, so resulting in agglutination of red blood cells, even in the presence of blood which had been treated with anticoagulants, such as heparin.

A study performed by Pusateri et al. (2003) compared the hemostatic properties of a chitosan gauze with a standard cotton gauze sponge. Liver injuries were induced in swine and the dressings were applied 30 seconds later. In the chitosan-treated gauze group, the blood loss was significantly reduced to approximately 264 ml, whereas in the group treated with the cotton gauze dressing, the blood loss was significantly greater, 2,879 ml. Consequently, the marked reduction of blood loss by the chitosan group resulted in a much higher survival rate for the swine thus treated in that seven of the eight swine in the chitosan-treated group survived, only two animals survived in the gauze-treated group.

Studies demonstrating the hemostatic capability of chitosan treated dressings warranted the comparison between the chitosan dressings and commercial collagen sponges and such a study was reported by Wang et al. (2006).

Wang et al. reported that in comparative studies using a rabbit cervical vein wound that the total amount of bleeding from the injured veins until hemostasis was achieved was essentially the same for both chitosan and collagen. Except for certain differences in the mechanical qualities of the experiment, the two sponges behaved similarly with regard to hemostasis. For example, the chitosan sponges were much more flexible and resistant to breakage than the collagen sponges and the chitosan sponge was degraded in situ much more slowly than the chitosan sponges.

Rao and Sharma (1997) studied the safety and hemostatic potential of chitosan and reported that autoclaving was an applicable sterilizing method in that it caused the least decrease in tensile strength and affected the rate of hemolysis rather negligibly.

In addition, they reported that sterilization of the chitosan with glutaraldehyde did in no way affect the maximum tensile strength of the chitosan. In vivo toxicity tests showed that the material was nontoxic and that sterilized films of the chitosan were free of pyrogen.

The two workers reported that the hemostatic capability of chitosan was independent of the classical coagulation methods and appears to be an interaction between the cell membrane of the erythrocytes and chitosan. This observation was reported in greater detail in an attempt to explain how chitosan was acting as a hemostat. It is well known in the profession that the negative surface charge on red blood cells is principally due to the presence of neuraminic acid residues on the cell membranes. By removing the neuraminic acid with neuraminidase, it has been observed that chitosan would not then cause gelling of the red blood cells; it is therefore to be concluded that the gelling of the red blood cells is due to the interaction between the positively charged chitosan polymer with the neuraminic acid present on the surface of the red blood cells which provide a strong negative charge.

The hemostatic properties of chitosan have also been extensively reported by Malette et al. (1983).

Mi, F. L. et al., (2002) reported on a chitosan-containing dressing which was composed of two layers in which one of the layers contained silver sulfadiazine. The principal role of the chitosan was to act as a delivery system for silver sulfadiazine in which the layer containing the chitosan was to regulate the release of the silver sulfadiazine. The dressing was not designed and had no characteristics to affect the coagulation of the bleeding wound.

Khan and Peh studied the effect of different chitosan films and a non-chitosan commercial dressing with regard to the rate of healing and the ease of film removal from punch biopsy wounds in rats (2003). No studies were done nor were any preparations of chitosan made that were designed to assess the effectiveness of the chitosan to coagulate blood and/or stop bleeding.

The toxicity of chitosan when administered orally, as well as intravenously to experimental animals, has been shown to be extremely low. Thus, the LD₅₀ is in excess of 16 g/Kg in mice (See the 18^{th} Edition of Taber's Cyclopedic Medical Dictionary).

The use of chitosan in various pharmacological preparations has been described by Felt et al., 1998. Chitosan dressings prepared for humans have been in clinical use for the treatment of partial and full thickness dermal ulcers, leg ulcers, superficial wounds, abrasions, burns, and donor sites. Such chitosan dressings manufactured by the 3M Corporation under their trade name Tegasorb™ (Illum, 1998; McRight, 1998).

The antimicrobial properties of chitosan have been reported against a number of microorganisms and probably are due to the properties of chitosan in acting as a coagulant of the microbial cells due to the strong positive charge of the chitosan molecule (Muzzarelli et al. 1988; Muzzarelli et al. 1990).

Soerens et al. (U.S. Pat. No. 6,967,261) reported the composition of a bandage that could be used for acute wounds or minor wounds in which a multiple layered dressing included a first layer for curbing the wound site, a second layer which was placed over the bottom surface of the first layer for absorbing exudates and the second layer which included a poly (ethyleneoxide)-based compound as well as a chitosan-based compound. A third layer situated over the second layer, where the third layer was composed of a perforated film in which at least one antimicrobial agent was included in the bandage.

Tonegawa et al. (2003) reported on the use of a chitosan-containing composition which together with the additions of horseradish and hydrogen peroxide enhanced the removal of phenols from water. Chitosan in this product was solely concerned with its ability to eliminate colored reaction products, but the composition including horseradish and polyethylene glycol as well as activated carbon which were not designed nor could they be used for the preparation of a dressing to be used on humans or animals for the treatment of cuts or other bleeding lesions.

### DESCRIPTION OF THE PREFERRED EMBODIMENTS

Since many wounds may bleed and circumstances may require a quick hemostasis of such wounds, it might be useful to have a chitosan dressing prepared in the form of a gel that can be dispensed quickly from a tube and that such gel could be applied directly into the wound, as made necessary by the length and breadth of the wound that was bleeding. In addition, if the gel were prepared in the form of a foamed or porous gel, this would enhance the absorption of blood into the chitosan dressing and thereby speed up the interaction of the chitosan with the blood thus effecting a more immediate hemostasis.

Such a porous or foamed gelation of a chitosan product can be achieved by taking advantage of the highly acidic nature of an acid that is utilized to dissolve the chitosan such as acetic acid by adding a salt such as sodium bicarbonate which will react with the acid and thereby produce a foaming within the gel that is so prepared. The preparation of such a composition is provided in Example 1.

### EXAMPLE 1

Chitosan, in an amount of 29 grams having a 200,000 centepoise (cps) viscosity was added to 550 ml of 8% acetic acid prepared by appropriately diluting glacial acetic acid with deionized or distilled water. Sodium bicarbonate in the amount of 19.2 grams is added to the chitosan-acetic acid mixture while stirring to produce a foam within the chitosan gel. Should it be desirable to enhance the amount of foaming of the chitosan gel thus prepared, it is possible to add approximately 1.5 to 2.0 grams of lauryl sulphate to the mixture while stirring which will increase the amount of foam that is entrapped in the chitosan gel.

Chitosan foamed gel, thus prepared, can be transferred to dispensing tubes and the bottom sealed so that the product may be sterilized by appropriate sterilization means and be provided to healthcare institutions that need only squeeze the gel onto a bleeding wound through the orifice of such a contained tube. Alternatively, the foamed chitosan composition described in Example 1 may be layered on a surface and dried at an appropriate temperature to provide a dressing that can be applied to a bleeding wound.

### EXAMPLE 2

If desired, the chitosan gel as prepared in Example 1 can be prepared as a gel which is layered onto a suitable backing, such as polyurethane, polyester, or cotton backing and dried at 125-130° F. for 8-10 hours so resulting in a dressing which has a backing to it. The preparation of the gel prior to layering onto the backing may have the following composition:

| | | |
|---|---|---|
| chitosan - 200,000 cps | 29 | grams |
| acetic acid 8% | 550 | ml |
| Stir and mix. | | |
| sodium bicarbonate | 19.2 | grams |
| Stir and mix. | | |
| lauryl sulphate | 1.5 | grams |
| glycerin | 10 | ml |
| propylene glycol | 5 | ml |
| polyethylene | 1 | gram |
| Stir to mix | | |

The polyethylene may be optional depending upon the viscosity of the final product that is desired to be layered onto a suitable backing or depending upon the viscosity of the final product that is required to fill a dispensing tube from which the foamed chitosan composition may be dispensed into and onto a bleeding wound.

### EXAMPLE 3

Properties of the chitosan gel as prepared in Examples 1 and 2 can have its softness and pliability enhanced by adding sodium alginate and an amount of calcium gluconate and Tween 80, the ingredients of which would make the chitosan gel more pliable when layered on a suitable backing and dried for use on wounds that require a hemostatic agent. Such a composition could be:

| | | |
|---|---|---|
| chitosan - 200,000 cps | 29 | grams |
| acetic acid 8% | 550 | ml |
| Stir and mix. | | |
| sodium bicarbonate | 19.2 | grams |
| Stir and mix. | | |
| lauryl sulphate | 1.5 | grams |
| glycerin | 10 | ml |
| propylene glycol | 5 | ml |
| polyethylene | 1 | gram |
| Stir to mix. | | |
| sodium alginate | 7 | grams |
| calcium gluconate | 2 | grams |
| Tween 80 ®, Atlas Chemical Industries, Inc. | 9 | ml |
| (polyoxethylenesorbitan monooleate) | | |

### EXAMPLE 4

Should it be desirable to add an ingredient to the chitosan composition that, in addition to retaining its hemostatic properties, would make it possible to provide a chemotactic agent that might be necessary if a wound is infected, such a compound has been described in many documents and maltodextrin is such a suitable compound, which when added to the composition set forth in example 1 or in Example 2 above, would provide a chemotactic activity as has been recorded in documents by Silvetti (1981), Silvetti (1993), Silvetti (1987), Braun et al. (1992), and Schwartz (1987). The proposed maltodextrin to be utilized in the preparation of the gel and dressing compositions described herein is substantially equivalent in intended use and composition as described by Scherr in the United States Food and Drug Administration 510(k) K011618.

Additional reports describing the use of maltodextrin and related starch hydrolysates including clinical and laboratory studies have also been reported in detail in the following patents:
U.S. Pat. No. 4,889,844 issued Dec. 26, 1989,
U.S. Pat. No. 4,414,202 issued Nov. 8, 1983,
U.S. Pat. No. 6,046,160 issued Apr. 4, 2000,
U.S. Pat. No. 5,171,065 issued Jan. 5, 1993

Such a formulation may be:

| | | |
|---|---|---|
| chitosan - 200,000 cps | 29 | grams |
| acetic acid 8% | 550 | ml |
| Stir and mix. | | |
| sodium bicarbonate | 19.2 | grams |
| Stir and mix. | | |
| lauryl sulphate | 1.5 | grams |
| glycerin | 10 | ml |
| propylene glycol | 5 | ml |
| polyethylene | 1 | gram |
| Stir to mix. | | |
| sodium alginate | 7 | grams |
| calcium gluconate | 2 | grams |
| Tween 80 ®, Atlas Chemical Industries, Inc. | 9 | ml |
| (polyoxethylenesorbitan monooleate) | | |
| Maltodextrin | 5 | grams |
| (dextrose equivalent of 10-12, mw 3000 daltons) | | |

### EXAMPLE 5

Should it be desirable to enhance the antimicrobial activity of the chitosan gel as prepared in Examples 1, 2, 3, and 4, it is possible to add an antimicrobial agent to the chitosan composition without deviating from the essential capability of the chitosan to act as a hemostatic agent. Although chitosan has been acknowledged as having some antimicrobial activity, this may in part be due to its ability to agglomerate various cell particles and the addition of a more active antimicrobial agent may be necessary in bleeding wounds which are highly susceptible to infection.

Antimicrobial agents that may be added to the chitosan composition or other medicaments and/or drugs that may be utilized within the chitosan composition and thus be delivered by means of this invention into a wound site, but are not limited to this invention may include:

Analgesics narcotic and non-narcotic (e.g. Morphine, Hydromorphine, Pentazocine, APAP)

Anti-inflammatory drugs steroidal (e.g. Hydrocortisone)

Anti-inflammatory drugs, non-steroidal (e.g. Pioroxicam, Naproxen, Diclofenac, Ketoprofen, Ibuprofen)

Local anesthetics (e.g. Lidocaine, Pramoxine, Benzocaine)

Steroids (e.g. Testosterone, Estradiol, Progesterone and its conjugates

Antifungal (e.G. Miconazole, Econazole, Terconazole)

Antiviral (e.g. Acyclover, Behenyl Alcohol)

Silver containing compounds such as silver alginate, silver nitrate.

The above descriptions and examples illustrate particular constructions including the preferred embodiments of the chitosan dressings. However, the invention is not limited to the precise constructions described herein, but rather, all modifications and improvements thereof encompassed within the scope of the invention.

The chitosan utilized in the examples described herein utilized a chitosan of a 200,000 centepoise (cps) and in a weight of that composition shown at 29 grams. It is clear that other chitosans of varying concentrations and varying centepoise viscosity may also be utilized without deviating from the novelty of the revelations contained in this patent as long as the chitosan is of a concentration and viscosity that can be reasonably used as a gel and perform the hemostatic activity required within the scope of the invention.

The acetic acid utilized in the examples set forth herein utilizes an 8% solution and an amount of the composition of 550 ml. It is clear that, depending upon the chitosan used, the acetic acid may vary in amount and also in concentration without in any way deviating from the capability of the chitosan to act as a hemostatic agent in the compositions as set forth in the examples cited herein. The use of sodium bicarbonate to react with the acetic acid makes it possible to achieve a foam composition and such foam resulting in an enhanced surface area of the chitosan gel would have the ability to enhance the absorption of blood as well as increase the release of chitosan ions into the blood to achieve the hemostatic capability of the chitosan that is desirable for the dressing thus prepared.

Although, sodium bicarbonate has been utilized in the examples given herein, other alkaline salts, such as sodium carbonate may also be used which would react with the acetic acid of the chitosan composition to provide a foam, other methods may be utilized to provide the foamed chitosan product without deviating from the hemostatic capability of the chitosan desirable for the dressing thus prepared. For example, vigorous stirring of the chitosan composition, depending upon its viscosity, will entrap air bubbles to provide the foam desirable for the chitosan product. Alternatively, stirring of the chitosan acidified composition while desirable gases are permitted to flow into the chitosan composition will also serve to entrap a foam within the chitosan composition.

The addition of a relatively small amount of lauryl sulphate is provided to enhance the foam that results from the addition of a bicarbonate or carbonate sodium salt with the acetic acid since lauryl sulphate is well known in the profession in enhancing the entrapment of air when stirring in providing a foam product. Depending upon the concentration of chitosan, acetic acid, and the sodium bicarbonate, the addition of lauryl sulphate may or may not be an additional attribute if the foam provided by the particular viscosity of chitosan and carbonate or bicarbonate of sodium utilized is sufficient in and of themselves.

Note that in the examples cited herein, the effervescent compound that reacts with the water soluble dilute acetic acid with the resultant evolution of gases which become entrapped in the formation of the gel network is sodium bicarbonate. Other water soluble effervescent compounds may be utilized and other acids may be utilized to produce the evolution of gases which become entrapped in the chitosan gel network without deviating from the novelty of the invention described herein. Thus, various water insoluble metal salts than can react with water soluble acids are calcium carbonate, calcium phosphate dibasic, barium carbonate, calcium phosphate dibasic, barium carbonate, or zinc carbonate.

The use of glycerin, propylene glycol, and polyethylene provide a soft texture of the gel composition and are well known in the profession; their attributes of which have been recorded in the past such as in U.S. Pat. No. 6,696,077 B2. The concentrations of glycerin, propylene glycol and polyethylene are provided here in order to effect the texture and softness of the gel dressing as may be prepared as described herein, but it is clear that depending upon the chitosan used, as well as its concentration and viscosity, other concentrations of the glycerin, propylene glycol and polyethylene may be provided without in any way deviating from the hemostatic activity for which the dressing has been principally compounded. It is well known in the profession that various glycols will act as plasticizers and may be used to improve the flexibility of chitosan gels or fibers. The plasticizer that we have principally used in the examples described herein has been glycerin because of its low cost, low toxicity, and ready availability. It is clear, however, that other plasticizers may be utilized such as propylene glycol, or ethylene glycol without deviating from the novelty of the invention described herein.

Example 3 provides the latitude to add sodium alginate and calcium gluconate as well as Tween 80 to the composition of Examples 1 and 2 in order to enhance the cohesiveness of the dressing when it is desired to layer the chitosan gel composition on a suitable backing. Although the alginate used in the examples described herein is sodium alginate, it is clear that other water soluble alginates may be utilized without deviating from the novelty of the invention described herein such as aqueous soluble ammonium alginate, or potassium alginate. In the examples cited herein, calcium gluconate has been utilized to provide the calcium ion, which precipitates the insoluble calcium alginate which serves to entrap into the calcium alginate matrix, other components and wherein the calcium alginate enhances the cohesiveness of the chitosan dressing, it is clear that other salts may be utilized to precipitate the alginate. Such salts such as alkaline earth metal salts, alkali metal salts, transition metal salts, and mixtures thereof; in particular salts of calcium, barium, copper, magnesium, iron, zinc, aluminum, manganese, strontium, chromium, or silver. These insoluble alginates may readily be utilized to precipitate the cohesive alginate mixtures described in the examples provided herein without deviating from the essential merits of this invention. However, since the alginate compositions are to be utilized in and on biological tissues, the particular salt utilized to precipitate the alginate should be dictated by any restraints of toxicity or other untoward reactions that might result from their use for the preparation of bandages, dressings, or surgical products as described herein. Again, it is noted that the sodium alginate, calcium gluconate, and Tween 80 have been used in previous patents, such as U.S. Pat. No. 6,696,077 B2, are well known in the profession, and may be utilized here to enhance the pliability that may be desirable in the treatment of wounds which are bleeding to an extent that requires the use of a hemostat such as the chitosan composition provided herein.

The use of a surface active agent such as Tween 80 is utilized primarily to effect a dispersion between the aqueous and non-aqueous miscible components as well as to achieve a homogeneity with the component agents.

Such surface active agents are also utilized in order to improve the wetting of a medical dressing or bandage in the event that a wound may be exudating, and the enhanced wicking in such a bandage or medical dressing serves to quickly absorb any blood or serum from a wound into the dressing. Other surface active agents, such as the Na salt of dodecyl SO₄ (sodium lauryl sulfate) or a member of the group of Tweens: Tween 20, polyoxyethylene sorbitan monolaurate; Tween 40, polyoxyethylene sorbitan monopalmitate, or Tween 85, polyoxyethylene sorbitan trioleate may be incorporated into the chitosan composition without deviating from the novelty of the invention described herein.

Since wounds that have been exposed to air and may be bleeding are particularly susceptible to infection, an additional option of an agent which is a chemotactic agent can be utilized such as maltodextrin. Maltodextrin acts as a chemotactic agent for white blood cells to remove either living or dead microorganisms from a bleeding site or site of infection and have been described in detail in patents and other references cited here in Example 4.

I claim:

1. A process for making a foamed chitosan composition to be utilized in the preparation of wound dressings requiring a hemostatic product comprising the steps of:
(I) making an acidic solution of chitosan utilizing an aqueous soluble acid;
(II) while allowing the composition of the acid solution and the chitosan to be mixed, adding a gaseous foam-forming or effervescent compound(s) to react with the acid.

2. The process of claim 1 wherein the said effervescent compound is selected from the group consisting of the alkali metal carbonates.

3. The process of claim 2 wherein said effervescent compound is sodium bicarbonate.

4. The process of any preceding claim wherein said effervescent compound is sodium carbonate, zinc carbonate, calcium carbonate, or barium carbonate.

5. The process of any preceding claim wherein said aqueous soluble acid is selected from a group consisting of acetic, lactic, malic, gluconic, and ascorbic acids.

6. The process of any preceding claim wherein a medicament is added to the foamed chitosan composition.

7. The process of claim 6 wherein said medicament is selected from the group consisting of collagen, maltodextrin, antibiotics, antibacterial agents, anti-inflammatory agents, ascorbic acid, amino acids, and mixtures thereof.

8. The process of any preceding claim wherein the foaming is enhanced by the addition of lauryl sulfate.

9. The process of any preceding claim wherein a plasticizer and a surface active agent are added to said mixture.

10. The process of claim 9 wherein said plasticizer is selected from a group consisting of glycerin, propylene glycol, ethylene glycol, and polyethylene glycol or mixtures thereof.

11. The process of any preceding claim wherein a surface active agent is added to the foamed chitosan composition.

12. The process of claim 11 wherein said surface active agent is sodium lauryl sulfate, pluronic L64, Tween 80, Tween 20, Tween 40, Tween 85.

13. The process of any preceding claim wherein a water soluble alginate is added to the foamed chitosan composition and said water soluble alginate is selected from a group consisting of ammonium, potassium, and sodium salts of alginate, or mixtures thereof.

14. The process of claim 13 wherein said cationic metal ion is utilized to render the water soluble alginate, water insoluble and wherein said metal ion is derived from salts selected from the group consisting of alkaline earth metal salts, alkali metal salts, transition metal salts, and mixtures thereof.

15. The process of claim 14 wherein said cationic metal ion is selected from the group consisting of calcium, barium, copper, magnesium, iron (ferric and ferrous), zinc, aluminum, manganese, strontium, silver, chromium, and mixtures thereof.

16. The process of any preceding claim wherein said foamed chitosan composition may be used to fill a dispensing tube from which the foamed chitosan composition may be dispensed into and onto a bleeding wound.

17. The process of any preceding claim wherein said foamed chitosan composition may be poured onto a surface that will permit the evaporation of water so resulting in a sheet of a foamed chitosan composition.

18. The process of claim 17 wherein the surface on to which the composite chitosan mixture is poured is a fibrous cloth.

19. The process of claim 18 wherein said fibrous cloth is selected from cloths prepared from cotton, polyester, wool, nylon, rayon, or mixtures thereof.

20. A wound dressing suitable for direct application to a wound comprising a sterile backing having layered thereon a foamed chitosan composition produced by a process according to any of the previous claims.

21. A foamed chitosan composition suitable for use as a wound dressing characterized in that said composition prepared by any of the previous claims may be used to fill a dispensing tube from which the foamed chitosan composition may be dispensed into and onto a bleeding wound.

22. A foamed chitosan composition suitable for use in a wound dressing.

23. A foamed chitosan composition suitable for use as a hemostat.
